(19) United States
(12) Patent Application Publication
(10) Pub. No.: US 2026/0242322 A1
(43) Pub. Date: Aug. 20, 2026

(54) THERMOLYTIC FRAGMENTATION OF SUGARS

(71) Applicant: Topsoe A/S, Kgs. Lyngby (DK)

(72) Inventors: Morten Boberg LARSEN, Smørum (DK); Max THORHAUGE, Herlev (DK); Christian Mårup OSMUNDSEN, Gentofte (DK)

(73) Assignee: Topsoe A/S, Kgs. Lyngby (DK)

(21) Appl. No.: 18/995,033

(22) PCT Filed: Aug. 28, 2023
(86) PCT No.: PCT/EP2023/073517

§ 371 Date: Jan. 15, 2025

(30) Foreign Application Priority Data
Aug. 29, 2022 (EP) 22192643.9

(51) Int. Cl. C07C 45/51; B01J 8/18; C07C 45/78

(52) U.S. Cl. CPC C07C 45/511; B01J 8/18; C07C 45/78

(57) ABSTRACT

The present invention relates to a method for thermolytic fragmentation of a sugar into C1-C3 oxygenates, comprising separating solids from the fragmentation product, wherein the temperature of the fragmentation product at an outlet of the reactor is at least 250° C., and wherein the water content is at least 40 vol. %, based on all gas phase constituents. The present invention also relates to a system for performing the thermolytic fragmentation of a sugar into C1-C3 oxygenates. The method and the system are suitable for industrial scale production.

### FIELD OF THE INVENTION

The present invention relates to a method for thermolytic fragmentation of a sugar into C1-C3 oxygenates. The present invention also relates to a system for thermolytic fragmentation of a sugar into C1-C3 oxygenates. The method and the system are suitable for industrial scale production.

### BACKGROUND

Biomass is of particular interest as a raw material due to its potential for supplementing and possibly replacing petroleum as a feedstock for the preparation of commercial chemicals. In recent years, various technologies for exploiting biomass have been investigated. Carbohydrates represent a large fraction of biomass, and various strategies for their efficient use as a feedstock for the preparation of commercial chemicals are being established. These strategies include various fermentation-based processes, pyrolysis, and different processes employing catalysis such as hydrogenolysis, conversion via retro-aldol chemistry, and various acid catalysed dehydration processes.

Examples of chemicals produced from biomass include: small alcohols such as ethanol, propanol, and butanol, monomers such as ethylene glycol, ethylene, propylene, butadiene, isoprene, furandicarboxylic acid, succinic acid, lactic acid and lactide, acrylic acid, epichlorohydrin, and vinyl glycolic acid. Also included are diols such as 1,2-propanediol, 1,2-butanediol, 2,3-butanediol, 1,2-pentanediol, 1,2-hexanediol, and sorbitol, glycerol, glycolaldehyde, pyruvaldehyde, and levulinic acid. Other products such as substitute natural gas, synthesis gas, and biofuels are also included.

Within the field of pyrolysis, efforts have been focused on using feedstocks based on solid biomass and other lignocellulosic materials for producing the above chemicals.

Some efforts have been made to use sugars as feedstock for producing food browning materials, which comprise a large amount of glycolaldehyde (also termed hydroxyacetaldehyde) as the key browning agent.

Fluidised bed reactors comprising heat carrying particles are used for processing a variety of feedstocks. They can be operated in a number of different fluidisation regimens. The preferred regime is selected depending on the feedstock in question and the desired chemistry to be obtained, which gives rise to a large number of different reactor configurations for fluidised bed reactors.

For conversion of biomass into bio-oil by pyrolysis, several reactor configurations have been investigated, such as e.g. dense phase (i.e. bubbling fluidised bed) and dilute phase (i.e. riser) reactors, as well as radically different reactor types, such as ablative pyrolysis reactors.

Examples of prior art disclosing the use of fluidised bed reactors include U.S. Pat. No. 5,397,582 (Underwood), U.S. Pat. No. 7,094,932 (Majerski), WO 2014/131764, WO 2012/115754, U.S. Pat. No. 5,302,280 (Lomas), and WO 2017/216311 (Larsen et al.).

There remains a need for improved methods and systems for thermolytic fragmentation of a sugar into C1-C3 oxygenates, in particular which are suitable for industrial scale production.

### SUMMARY OF THE INVENTION

According to an aspect of the present invention, there is provided a process for thermolytic fragmentation of a sugar into C1-C3 oxygenates, the process comprising the steps of:
a) providing an aqueous feedstock solution comprising the sugar;
b) providing a fluidised bed fragmentation reactor for thermolytically fragmenting the sugar and comprising fluidisable heat carrying particles;
c) introducing the feedstock solution into the reactor to thermolytically fragment the sugar to provide a fragmentation product comprising the C1-C3 oxygenates; and
d) separating solids from the fragmentation product, wherein the solids comprise solids selected from the fluidisable heat carrying particles, fragments of the fluidisable heat carrying particles, by-products from the thermolytic fragmentation of the feedstock solution, and mixtures thereof;
wherein the temperature of the fragmentation product at an outlet of the reactor is at least 250° C., such as at least 300° C., at least 350° C., at least 400° C., or at least 450° C., and wherein the water content is at least 40 vol. %, based on all gas phase constituents.

According to another aspect of the present invention, there is provided a process for thermolytic fragmentation of a sugar into C1-C3 oxygenates, the process comprising the steps of:
a) providing an aqueous feedstock solution comprising the sugar;
b) providing a fluidised bed fragmentation reactor for thermolytically fragmenting the sugar and comprising fluidisable heat carrying particles;
c) introducing the feedstock solution into the reactor to thermolytically fragment the sugar to provide a fragmentation product comprising the C1-C3 oxygenates; and
d) separating solids from the fragmentation product, wherein the solids comprise solids selected from the fluidisable heat carrying particles, fragments of the fluidisable heat carrying particles, by-products from the thermolytic fragmentation of the feedstock solution, and mixtures thereof;
wherein the temperature of the fragmentation product at an outlet of the reactor is at least 250° C., such as at least 300° C., at least 350° C., at least 400° C., or at least 450° C., and wherein the partial pressure of steam is greater than 650 mbar.

The present inventors have investigated means for providing improved processes and systems for thermolytic fragmentation of a sugar into C1-C3 oxygenates. Such processes and systems typically employ a fluidised bed fragmentation reactor for thermolytically fragmenting a sugar to provide a fragmentation product comprising the C1-C3 oxygenates.

During such investigations, the present inventors observed problems in the operation of equipment downstream of the reactor. The present inventors surprisingly discovered the presence of material adhered to the equipment. In some instances, this material resulted in obstruction or blockage in the equipment.

Upon making such discoveries, the present inventors conducted experiments to characterise the material, and identified that such material comprised solids selected from fluidisable heat carrying particles (transported from the reactor), fragments of the fluidisable heat carrying particles (transported from the reactor), by-products from the thermolytic fragmentation of the feedstock solution, and mixtures thereof.

The present inventors tested various means (e.g. a physical separation device such as a filter) for reducing the amount of the solids transported downstream of the reactor. Whilst such means initially helped to achieve this, the present inventors further discovered that the solids deposited onto such means (e.g. as “filter cake”) in an increasing amount as a function of time (reactor run time). So as to facilitate effective process operation, the solids eventually were removed. However, removal of the solids by replacement of the means involves shutting down the process, resulting in significant downtime which is inefficient. Where the means comprised a filter, the present inventors also attempted to remove the solids from the filter by “back-pulsing” or “back-blowing” techniques, e.g. which involved directing air in a reverse direction through the filter. However, these techniques were not effective because the solids remained adhered to the filter.

With the above problems in mind, the present inventors surprisingly discovered that the presence of water in an amount of at least 40 vol. % based on all gas phase constituents resulted in an improved performance of equipment downstream of the reactor. In particular, such a water content resulted in reduced obstruction and/or blockage in equipment downstream of the reactor. Furthermore, where a physical separation device such a filter arranged downstream of the reactor was employed, the present inventors surprisingly also discovered that such a water content reduced the build-up of the solids on the physical separation device.

Without being bound by theory, it is hypothesised that such a water content reduces the amount of the solids that are transported downstream of the reactor and/or reduces the propensity of the solids to form and/or reduces the propensity of the solids to obstruct or block the equipment downstream of the reactor. Moreover, when a physical separation device such a filter arranged downstream of the reactor is employed, it is further hypothesised that such a water content reduces the amount of the solids that reach the physical separation device and/or reduces the propensity of the solids to adhere to the physical separation device and/or reduces the propensity of the solids to obstruct or block the physical separation device.

In the present context, the terms “downstream” and “upstream” are with respect to the direction in which the components flow. For example, the outlet of the reactor is downstream of the point at which the feedstock solution is introduced into the reactor, given that components flow from the point at which the feedstock solution is introduced to the outlet of the reactor.

The water content based on all gas phase constituents can be readily calculated by those skilled in the art, e.g. using input flow data.

### a) the Feedstock Solution

The process comprises providing an aqueous feedstock solution comprising the sugar.

In one aspect, the total sugar content in the feedstock solution is at least 30 wt. %, such as at least 35 wt. %, at least 40 wt. %, at least 45 wt. %, at least 50 wt. %, at least 55 wt. %, at least 60 wt. %, at least 65 wt. %, at least 70 wt. %, at least 75 wt. %, at least 80 wt. %, at least 85 wt. %, at least 90 wt. %, or at least 95 wt. %, based on the total weight of the feedstock solution.

In one aspect, the total sugar content in the feedstock solution is no greater than 99.9 wt. %, such as no greater than 99.5 wt. %, no greater than 99 wt. %, no greater than 95 wt. %, no greater than 85 wt. %, or no greater than 80 wt. %, based on the total weight of the feedstock solution.

In one aspect, the total sugar content in the feedstock solution is from 30 to 99 wt. %, such as from 40 to 90 wt. %, or from 50 to 80 wt. %, based on the total weight of the feedstock solution.

In one aspect, the feedstock solution is a liquid.

In one aspect, the water content in the feedstock solution is at least 20 wt. %, such as at least 25 wt. %, at least 30 wt. %, at least 35 wt. %, at least 40 wt. %, at least 45 wt. %, at least 50 wt. %, based on the total weight of the feedstock solution.

In one aspect, the water content in the feedstock solution is no greater than 70 wt. %, such as no greater than 65 wt. %, no greater than 60 wt. %, no greater than 55 wt. %, no greater than 50 wt. %, or no greater than 45 wt. %, based on the total weight of the feedstock solution.

In one aspect, the total sugar content in the aqueous feedstock solution is from 20 to 70 wt. %, such as from 25 to 60 wt. %, or from 30 to 50 wt. %, based on the total weight of the feedstock solution.

In one aspect, the sugar is a carbohydrate comprising one or more C6 saccharide units and/or C5 saccharide units.

In one aspect, the sugar is a monosaccharide or a disaccharide.

In one aspect, the sugar is selected from sucrose, lactose, xylose, arabinose, ribose, mannose, tagatose, galactose, glucose and fructose.

In one aspect, the feedstock solution may comprise a sugar syrup.

In one aspect, the feedstock solution comprises more than one sugar.

Each sugar may be independently as described herein.

### b) Providing a Fluidised Bed Fragmentation Reactor for Thermolytically Fragmenting the Sugar and Comprising Fluidisable Heat Carrying Particles

The process comprises providing a fluidised bed fragmentation reactor for thermolytically fragmenting the sugar and comprising fluidisable heat carrying particles.

As will be understood by those skilled in the art, a fluidised bed reactor is a reactor which accommodates a bed of particles which may be fluidised, e.g. by a fluidisation gas which may be introduced at the base of the reactor. The velocity and physical properties of the fluidisation gas combined with the physical properties of the fluidisable particles may regulate the fluidisation state of the fluidisable particles within the bed. A dense bed/turbulent bed/bubbling bed has a superficial velocity of the fluidisation gas within the reactor of from 0.01 to 2 m/s. A fast bed (also termed a riser/transport reactor) may have a superficial velocity of fluidisation gas within the reactor of from 3 to 22 m/s. The exact velocity range is, however, dependent on the physical properties of the fluidisable particles and the fluidisation gas, and can be determined experimentally or calculated by those skilled in the art.

In one aspect, the fluidised bed fragmentation reactor is operative, in the bubbling fluidisation regime, the slugging fluidisation regime, the turbulent fluidisation regime, the fast fluidisation regime, and/or the pneumatic conveying regime.

In one aspect, the fluidised bed fragmentation reactor is operative in the fast fluidisation regime. Such a reactor is also termed a “riser type reactor”. Thus, in one aspect, the reactor is a riser type reactor.

In one aspect, the reactor comprises a riser.

In one aspect, the riser extends vertically.

In one aspect, the riser (e.g. a lower part of the riser) comprises a fluidisation gas inlet.

In one aspect, the riser (e.g. a lower part of the riser) comprises a fluidisable particle inlet.

In one aspect, the riser (e.g. a lower part of the riser) comprises a feedstock inlet.

In one aspect, the fluidisable particle inlet is provided downstream of the fluidisation gas inlet.

In one aspect, the feedstock solution inlet is provided downstream of the fluidisable particle inlet.

In one aspect, the fluidisable heat carrying particles form a dense phase fluidised bed in a zone between the fluidisable particle inlet and the feedstock solution inlet.

In one aspect, the riser (e.g. a lower part of the riser) comprises a fluidisable particle and fluidisation gas inlet. In this way, the fluidisable heat carrying particles and the fluidisation gas can be introduced into the reactor via a common inlet, e.g. sequentially or at the same time.

In one aspect, the feedstock solution inlet is provided downstream of the fluidisable particle and fluidisation gas inlet.

In one aspect, the method comprises introducing fluidisable heat carrying particles into the reactor.

In one aspect, the method comprises introducing fluidisable particles into the reactor via the fluidisable particle inlet.

In one aspect, the method comprises introducing fluidisable particles into the reactor via the fluidisable particle and fluidisation gas inlet.

In one aspect, the temperature of the fluidisable heat carrying particles within the reactor is at least 250° C., such as at least 300° C., at least 350° C., at least 400° C., at least 450° C., at least 500° C., at least 550° C., at least 600° C., or at least 650° C.

In one aspect, the temperature of the fluidisable heat carrying particles within the reactor is no greater than 800° C., such as no greater than 700° C., or no greater than 650° C.

In one aspect, the temperature of the fluidisable heat carrying particles within the reactor is from 400 to 800° C., such as from 500 to 800° C., from 450 to 650° C., or from 550 to 650° C.

In one aspect, the temperature of the fluidisable heat carrying particles at the fluidisable particle inlet (or the fluidisable particle and fluidisation gas inlet) is at least 250° C., such as at least 300° C., at least 350° C., at least 400° C., at least 450° C., at least 500° C., at least 550° C., at least 600° C., or at least 650° C.

In one aspect, the temperature of the fluidisable heat carrying particles at the fluidisable particle inlet (or the fluidisable particle and fluidisation gas inlet) is no greater than 800° C., such as no greater than 700° C., or no greater than 650° C.

In one aspect, the temperature of the fluidisable heat carrying particles at the fluidisable particle inlet (or the fluidisable particle and fluidisation gas inlet) is from 400 to 800° C., such as from 500 to 800° C., from 450 to 650° C., or from 550 to 650° C.

In one aspect, the pressure at the outlet of the reactor is at least 0.9 bara, such as at least 1 bara, at least 1.2 bara, at least 1.4 bara, at least 1.6 bara, at least 1.7 bara, at least 1.8 bara, at least 2 bara, or at least 2.2 bara.

In one aspect, the pressure at the outlet of the reactor is no greater than 8 bara, such as no greater than 6 bara, no greater than 5 bara, no greater than 4 bara, no greater than 3.5 bara, no greater than 3 bara, no greater than 2.9 bara, or no greater than 2.8 bara.

In one aspect, the pressure at the outlet of the reactor is from 0.9 to 8 bara, such as from 1 to 6 bara, from 1.1 to 5 bara, from 1.2 to 4 bara, from 1.5 to 3 bara, from 1.6 to 2.9 bara, from 1.7 to 2.8 bara, from 1.8 to 2.8 bara, from 2 to 2.8 bara, or from 2.2 to 2.8 bara.

In one aspect, the fluidisable heat carrying particles are selected from sand, mullite, silica, glass, alumina, silica-alumina, steel, and silicon carbide.

In one aspect, the mean particle size of the fluidisable heat carrying particles is from 20 to 400 μm, such as from 20 to 300 μm, or from 20 to 200 μm. The particle size distribution can be measured by laser diffraction spectroscopy, microscopy, electrical zone sensing. The mean particle size can be subsequently calculated from the particle size distribution. The mean particle size may be the sauter mean particle size. Those skilled in the art will appreciate that other mean particles sizes are envisaged.

As will be appreciated by those skilled in the art, the flow of the fluidisable heat carrying particles within the reactor may be adjusted relative to the flow of the feedstock solution so as to provide the desired amount of heat to the feedstock solution. Fluidisable heat carrying particles with a high heat capacity may require a lower mass flow rate than fluidisable heat carrying particles with a lower heat capacity.

c) Introducing the Feedstock Solution into the Reactor to Thermolytically Fragment the Sugar to Provide a Fragmentation Product Comprising the C1-C3 Oxygenates

The method comprises introducing the feedstock solution into the reactor to thermolytically fragment the sugar to provide a fragmentation product comprising the C1-C3 oxygenates.

The thermolytic fragmentation of sugar is an endothermic reaction, mainly due to evaporation of any liquid in the feedstock solution.

In one aspect, the temperature difference of the fluidisable particles between the fluidisable particle inlet (or the fluidisable particle and fluidisation gas inlet) of the fragmentation reactor and the fluidisable particle outlet of the fragmentation reactor is within the range of from 10 to 600° C., such as from 50 to 250° C., or from 50 to 150° C.

In one aspect, in step c) the feedstock solution is introduced into the reactor at a rate of at least 1000 kg/h (kilogrammes per hour). In one aspect, in step c) the feedstock solution is introduced into the reactor at a rate of at least 2000 kg/h. In one aspect, in step c) the feedstock solution is introduced into the reactor at a rate of at least 5000 kg/h. In one aspect, in step c) the feedstock solution is introduced into the reactor at a rate of at least 10000 kg/h. In one aspect, in step c) the feedstock solution is introduced into the reactor at a rate of at least 15000 kg/h. In one aspect, in step c) the feedstock solution is introduced into the reactor at a rate of at least 20000 kg/h.

There may be no upper limit as to the rate at which feedstock solution is introduced into the reactor in step c). In one aspect, in step c) the feedstock solution is introduced into the reactor at a rate of no greater than 1000000 kg/h. In one aspect, in step c) the feedstock solution is introduced into the reactor at a rate of no greater than 100000 kg/h.

### Fluidisation Gas

In one aspect, the process comprises introducing a fluidisation gas into the reactor.

As will be understood by those skilled in the art, the fluidisation gas fluidises the fluidisable heat carrying particles in the reactor.

In one aspect, the process comprises introducing a fluidisation gas into the reactor via the fluidisation gas inlet.

In one aspect, the process comprises introducing a fluidisation gas into the reactor via the fluidisable particle and fluidisation gas inlet.

When the feedstock solution encounters the fluidisable heat carrying particles in the reactor, a vaporisation zone is formed in which the liquid evaporates and gaseous products are generated via sugar fragmentation. This results in an increase in the superficial velocity of the gas products thereby entraining the fluidisable heat carrying particles. Accordingly, downstream of the feedstock solution inlet, the fluidisable heat carrying particles and the feedstock solution form a fast bed above the vaporisation zone.

In one aspect, step c) comprises introducing the feedstock solution and the fluidisation gas into the reactor to thermolytically fragment the sugar to provide the fragmentation product.

In one aspect, in step c) the feedstock solution is entrained in the fluidisation gas.

In one aspect, the fluidisation gas comprises water (e.g. steam).

In one aspect, the fluidisation gas consists essentially of water (e.g. steam).

In one aspect, the fluidisation gas consists of water (e.g. steam).

Herein, “water” and “steam” may be used interchangeably.

In one aspect, the water content in the fluidisation gas is at least 50 wt. %, such as at least 60 wt. %, at least 70 wt. %, at least 75 wt. %, at least 80 wt. %, at least 85 wt. %, at least 90 wt. %, at least 95 wt. %, or at least 99 wt. %, based on the total weight of the fluidisation gas.

In one aspect, the water content in the fluidisation gas is no greater than 90 wt. %, such as no greater than 95 wt. %, no greater than 99 wt. %, or no greater than 99.5 wt. %, based on the total weight of the fluidisation gas.

In one aspect, in step c) the fluidisation gas is introduced into the reactor at a rate of at least 100 kg/h. In one aspect, in step c) the fluidisation gas is introduced into the reactor at a rate of at least 500 kg/h. In one aspect, in step c) the fluidisation gas is introduced into the reactor at a rate of at least 1000 kg/h. In one aspect, in step c) the fluidisation gas is introduced into the reactor at a rate of at least 2000 kg/h. In one aspect, in step c) the fluidisation gas is introduced into the reactor at a rate of at least 4000 kg/h. In one aspect, in step c) the fluidisation gas is introduced into the reactor at a rate of at least 5000 kg/h.

There may be no upper limit as to the rate at which the fluidisation gas is introduced into the reactor in step c). In one aspect, in step c) the fluidisation gas is introduced into the reactor at a rate of no greater than 50000 kg/h. In one aspect, in step c) the fluidisation gas is introduced into the reactor at a rate of no greater than 20000 kg/h.

### Atomisation Gas

In one aspect, the process comprises introducing an atomisation gas into the reactor.

As will be understood by those skilled in the art, the atomisation gas is used to atomise the feedstock solution introduced into the reactor. The term atomising is meant to refer to turning a liquid into small droplets.

In one aspect, the atomisation gas and the feedstock solution are introduced into the reactor such that the feedstock solution is atomised by the atomisation gas.

In one aspect, the atomisation gas comprises water (e.g. steam).

In one aspect, the atomisation gas consists essentially of water (e.g. steam).

In one aspect, the atomisation gas consists of water (e.g. steam).

In one aspect, the water content in the atomisation gas is at least 50 wt. %, such as at least 60 wt. %, at least 70 wt. %, at least 75 wt. %, at least 80 wt. %, at least 85 wt. %, at least 90 wt. %, at least 95 wt. %, or at least 99 wt. %, based on the total weight of the atomisation gas.

In one aspect, the water content in the atomisation gas is no greater than 90 wt. %, such as no greater than 95 wt. %, no greater than 99 wt. %, or no greater than 99.5 wt. %, based on the total weight of the atomisation gas.

In one aspect, in step c) the atomisation gas is introduced into the reactor at a rate of at least 50 kg/h. In one aspect, in step c) the atomisation gas is introduced into the reactor at a rate of at least 100 kg/h. In one aspect, in step c) the atomisation gas is introduced into the reactor at a rate of at least 200 kg/h. In one aspect, in step c) the atomisation gas is introduced into the reactor at a rate of at least 500 kg/h. In one aspect, in step c) the atomisation gas is introduced into the reactor at a rate of at least 800 kg/h. In one aspect, in step c) the atomisation gas is introduced into the reactor at a rate of at least 1000 kg/h. In one aspect, in step c) the atomisation gas is introduced into the reactor at a rate of at least 1200 kg/h.

There may be no upper limit as to the rate at which the atomisation gas is introduced into the reactor in step c). In one aspect, in step c) the atomisation gas is introduced into the reactor at a rate of no greater than 20000 kg/h. In one aspect, in step c) the atomisation gas is introduced into the reactor at a rate of no greater than 10000 kg/h.

### Water Introduced

The process comprises introducing water into the reactor.

The water may be introduced in the form of one or both of gas (steam) and liquid. The water may be introduced in various different ways, and by one or more different means, including, for example, via the feedstock solution and/or via the fluidisation gas and/or via the atomisation gas).

In one aspect, water is introduced into the reactor via the fluidisation gas.

In one aspect, water is introduced into the reactor via the atomisation gas.

In one aspect, the mass ratio of the total amount of sugar introduced into the reactor to the total amount of water introduced into the reactor is at least 0.1:1, such as at least 0.2:1, at least 0.3:1, at least 0.4:1, at least 0.45:1, at least 0.5:1, at least 0.55:1, at least 0.6:1, at least 0.65:1, at least 0.7:1, at least 0.75:1, at least 0.8:1, at least 0.85:1, at least 0.9:1, or at least 0.95:1.

In one aspect, the mass ratio of the total amount of sugar introduced into the reactor to the total amount of water introduced into the reactor is no greater than 5:1, such as no greater than 4:1, no greater than 3.5:1, no greater than 3:1, no greater than 2.5:1, no greater than 2:1, no greater than 1.5:1, no greater than 1.4:1, no greater than 1.3:1, no greater than 1.25:1, no greater than 1.2:1, no greater than 1.15:1, no greater than 1.1:1, no greater than 1.05:1, or no greater than 1:1.

In one aspect, the mass ratio of the total amount of sugar introduced into the reactor to the total amount of water introduced into the reactor is from 0.2:1 to 5:1, such as from 0.25:1 to 4:1, from 0.25:1 to 3.5:1 from 0.25:1 to 2.2:1, from 0.3:1 to 1.8:1, from 0.4:1 to 1.75:1, from 0.55:1 to 1.5:1, from 0.6:1 to 1.3:1, from 0.65:1 to 1.2:1, from 0.7:1 to 1:1, or from 0.75:1 to 0.90:1.

In one aspect, the mass ratio of water introduced into the reactor via the feedstock solution to the total amount of water introduced into the reactor is at least 0.001:1, such as at least 0.005:1, at least 0.01:1, at least 0.02:1, at least 0.04:1, at least 0.05:1, at least 0.1:1, at least 0.15:1, at least 0.2:1, at least 0.25:1, or at least 0.3:1.

In one aspect, the mass ratio of water introduced into the reactor via the feedstock solution to the total amount of water introduced into the reactor is no greater than 5:1, such as no greater than 4:1, no greater than 3:1, no greater than 2:1, no greater than 1:1, no greater than 0.8:1, no greater than 0.6:1, no greater than 0.55:1, no greater than 0.5:1, no greater than 0.45:1, or no greater than 0.4:1.

In one aspect, the mass ratio of water introduced into the reactor via the feedstock solution to the total amount of water introduced into the reactor is from 0.2:1 to 3:1, such as from 0.04:1 to 4:1, from 0.05:1 to 2:1, from 0.1 to 1:1, from 0.15:1 to 0.6:1, from 0.2:1 to 0.5:1, from 0.25:1 to 0.45:1, or from 0.3:1 to 0.4:1.

### The Fragmentation Product

The fragmentation product comprises C1-C3 oxygenates resulting from the thermolytic fragmentation of the feedstock solution. The fragmentation product provided in step (c) is a crude product. The fragmentation product provided in step (c) comprises the solids.

In one aspect, in step c) the fragmentation product is a solids-dense fragmentation product.

In one aspect, the solids-dense fragmentation product comprises the C1-C3 oxygenates and the solids.

In one aspect, step d) comprises separating solids from the solids-dense fragmentation product to provide a solids-lean fragmentation product, wherein the solids comprise solids selected from the fluidisable heat carrying particles, fragments of the fluidisable heat carrying particles, by-products from the thermolytic fragmentation of the feedstock solution, and mixtures thereof.

In one aspect, the solids-lean fragmentation product comprises the C1-C3 oxygenates.

In one aspect, the solids-lean fragmentation product comprises a lower amount of the solids than the solids-dense fragmentation product.

The terms “lean” in “solids-lean” and “dense” in “solids-dense” herein are relative to each other.

In one aspect, the C1-C3 oxygenates comprise one, more, or each of formaldehyde (C1), glycolaldehyde (C2), glyoxal (C2), pyruvaldehyde (C3), and acetol (C3). For most uses, the C2 and the C3 oxygenates are most valuable.

In one aspect, the fragmentation product comprises a mixture of two or more of C1-C3 oxygenates. The mixture may be interchangeably referred to as a C1-C3 oxygenate mixture, a C1-C3 oxygenate product, or C1-C3 oxygenates.

In one aspect, the fragmentation product comprises glycolaldehyde.

In one aspect, the fragmentation product includes a major portion of glycolaldehyde.

In one aspect, the fragmentation product comprises glycolaldehyde in an amount of at least 10 wt. %, such as at least 20 wt. %, at least 30 wt. %, at least 40 wt. %, at least 50 wt. %, at least 60 wt. %, or at least 70 wt. %, based on the total weight of the fragmentation product.

In one aspect, the fragmentation product comprises one or more of glycolaldehyde or glyoxal in an amount of at least 10 wt. %, such as at least 20 wt. %, at least 30 wt. %, at least 40 wt. %, at least 50 wt. %, at least 60 wt. %, at least 70 wt. %, or at least 80 wt. %, based on the total weight of the fragmentation product.

In one aspect, the fragmentation product comprises one or more of pyruvaldehyde or acetol in an amount of at least 3 wt. %, such as at least 5 wt. %, or at least 7 wt. %, based on the total weight of the fragmentation product.

In one aspect, the total amount of C1-C3 oxygenates in the fragmentation product is no greater than 99 wt. %, such as no greater than 95 wt. %, or no greater than 90 wt. %, based on the total weight of the fragmentation product.

In one aspect, “based on the total weight of the fragmentation product” refers to the fragmentation product provided in step (c) (e.g. the solids-dense fragmentation product).

In one aspect, “based on the total weight of the fragmentation product” refers to the fragmentation product following step (d) (e.g. the solids-lean fragmentation product).

In one aspect, “based on the total weight of the fragmentation product” refers to the fragmentation product not including the solids.

### Water Content in Vol. %

In one aspect, the water content is based on all gas phase constituents at the outlet of the reactor.

In one aspect, the water content is based on all gas phase constituents within the reactor.

In one aspect, the water content is measured (e.g. using suitable instrumentation). Suitable instrumentation for measuring water content will be known to those skilled in the art.

In one aspect, the water content is calculated (e.g. theoretically determined). Suitable means of calculating water content will be known to those skilled in the art.

As explained in “d) separating solids from the fragmentation product” below, in one aspect, step d) comprises separating the solids from the fragmentation product using a physical separation device.

In one aspect, where a physical separation device (e.g. a filter) is used, the water content is based on all gas phase constituents at an inlet of the physical separation device (e.g. a filter).

In one aspect, the water content at least 45 vol. %, such as at least 50 vol. %, at least 55 vol. %, at least 60 vol. %, at least 65 vol. %, at least 70 vol. %, at least 75 vol. %, or at least 80 vol. % based on all gas phase constituents.

In one aspect, the water content at the outlet of the reactor is at least 45 vol. %, such as at least 50 vol. %, at least 55 vol. %, at least 60 vol. %, at least 65 vol. %, at least 70 vol. %, at least 75 vol. %, or at least 80 vol. %, based on all gas phase constituents at the outlet of the reactor.

In one aspect, the water content within the reactor is at least 45 vol. %, such as at least 50 vol. %, at least 55 vol. %, at least 60 vol. %, at least 65 vol. %, at least 70 vol. %, at least 75 vol. %, or at least 80 vol. %, based on all gas phase constituents within the reactor.

In one aspect, the water content at the inlet of the physical separation device (e.g. filter) is at least 45 vol. %, such as at least 50 vol. %, at least 55 vol. %, at least 60 vol. %, at least 65 vol. %, at least 70 vol. %, at least 75 vol. %, or at least 80 vol. %, based on all gas phase constituents at the inlet of the physical separation device.

In one aspect, the water content is no greater than 99 vol. %, such as no greater than 95 vol. %, no greater than 90 vol. %, no greater than 85 vol. %, or no greater than 80 vol. %, based on all gas phase constituents.

In one aspect, the water content at the outlet of the reactor is no greater than 99 vol. %, such as no greater than 95 vol. %, no greater than 90 vol. %, no greater than 85 vol. %, or no greater than 80 vol. %, based on all gas phase constituents at the outlet of the reactor.

In one aspect, the water content within the reactor is no greater than 99 vol. %, such as no greater than 95 vol. %, no greater than 90 vol. %, no greater than 85 vol. %, or no greater than 80 vol. %, based on all gas phase constituents within the reactor.

In one aspect, the water content at the inlet of the physical separation device (e.g. filter) is no greater than 99 vol. %, such as no greater than 95 vol. %, no greater than 90 vol. %, no greater than 85 vol. %, or no greater than 80 vol. %, based on all gas phase constituents at the inlet of the physical separation device.

In one aspect, the water content is from 45 to 99.5 vol. %, such as from 50 to 99 vol. %, from 55 to 97.5 vol. %, from 60 to 92.5 vol. %, from 65 to 90 vol. %, from 70 to 87.5 vol. %, or from 75 to 85 vol. %, based on all gas phase constituents.

In one aspect, the water content at the outlet of the reactor is from 45 to 99.5 vol. %, such as from 50 to 99 vol. %, from 55 to 97.5 vol. %, from 60 to 92.5 vol. %, from 65 to 90 vol. %, from 70 to 87.5 vol. %, or from 75 to 85 vol. %, based on all gas phase constituents at the outlet of the reactor.

In one aspect, the water content within the reactor is from 45 to 99.5 vol. %, such as from 50 to 99 vol. %, from 55 to 97.5 vol. %, from 60 to 92.5 vol. %, from 65 to 90 vol. %, from 70 to 87.5 vol. %, or from 75 to 85 vol. %, based on all gas phase constituents within the reactor.

In one aspect, the water content at the inlet of the physical separation device (e.g. filter) is from 45 to 99.5 vol. %, such as from 50 to 99 vol. %, from 55 to 97.5 vol. %, from 60 to 92.5 vol. %, from 65 to 90 vol. %, from 70 to 87.5 vol. %, or from 75 to 85 vol. %, based on all gas phase constituents at the inlet of the physical separation device.

### Partial Pressure

In one aspect, the partial pressure of steam is greater than 650 mbar.

The present inventors have also found that the presence of steam at a partial pressure of greater than 650 mbar helps to reduce the amount of such solids being transported downstream of the reactor and negatively affecting equipment.

In one aspect, the partial pressure of steam is at an outlet of the reactor.

In one aspect, the partial pressure of steam is within the reactor.

In one aspect, where a physical separation device (e.g. a filter) is used, the partial pressure is at the physical separation device (e.g. an inlet thereof).

As will be understood by those skilled in the art, the partial pressure of a gas may be defined by the below formula:

$${p}_{i} = {x}_{i} \times p$$

in which pᵢ is the partial pressure of a component in a gas mixture, xᵢ is the volume fraction of the component in the gas mixture, and p is the total pressure of the gas mixture.

In one aspect, the partial pressure is measured (e.g. using suitable instrumentation).

Suitable instrumentation for measuring partial pressure will be known to those skilled in the art.

In one aspect, the partial pressure is calculated (e.g. theoretically determined). Suitable means of calculating partial pressure will be known to those skilled in the art.

In one aspect, the partial pressure of steam is at least 700 mbar, such as at least 750 mbar, at least 800 mbar, at least 850 mbar, at least 900 mbar, at least 950 mbar, at least 1000 mbar, at least 1050 mbar, at least 1100 mbar, at least 1150 mbar, at least 1200 mbar, at least 1250 mbar, at least 1300 mbar, at least 1350 mbar, at least 1400 mbar, at least 1450 mbar, at least 1500 mbar, at least 1550 mbar, at least 1600 mbar, at least 1650 mbar, at least 1700 mbar, at least 1750 mbar, at least 1800 mbar, at least 1850 mbar, at least 1900 mbar, at least 1950 mbar, at least 2000 mbar, at least 2050 mbar, at least 2100 mbar, at least 2150 mbar, or at least 2200 mbar.

In one aspect, the partial pressure of steam at the outlet of the reactor is greater than 650 mbar. In one aspect, the partial pressure of steam at the outlet of the reactor is at least 700 mbar, such as at least 750 mbar, at least 800 mbar, at least 850 mbar, at least 900 mbar, at least 950 mbar, at least 1000 mbar, at least 1050 mbar, at least 1100 mbar, at least 1150 mbar, at least 1200 mbar, at least 1250 mbar, at least 1300 mbar, at least 1350 mbar, at least 1400 mbar, at least 1450 mbar, at least 1500 mbar, at least 1550 mbar, at least 1600 mbar, at least 1650 mbar, at least 1700 mbar, at least 1750 mbar, at least 1800 mbar, at least 1850 mbar, at least 1900 mbar, at least 1950 mbar, at least 2000 mbar, at least 2050 mbar, at least 2100 mbar, at least 2150 mbar, or at least 2200 mbar.

In one aspect, the partial pressure of steam within the reactor is greater than 650 mbar. In one aspect, as the partial pressure of steam within the reactor is at least 700 mbar, such as at least 750 mbar, at least 800 mbar, at least 850 mbar, at least 900 mbar, at least 950 mbar, at least 1000 mbar, at least 1050 mbar, at least 1100 mbar, at least 1150 mbar, at least 1200 mbar, at least 1250 mbar, at least 1300 mbar, at least 1350 mbar, at least 1400 mbar, at least 1450 mbar, at least 1500 mbar, at least 1550 mbar, at least 1600 mbar, at least 1650 mbar, at least 1700 mbar, at least 1750 mbar, at least 1800 mbar, at least 1850 mbar, at least 1900 mbar, at least 1950 mbar, at least 2000 mbar, at least 2050 mbar, at least 2100 mbar, at least 2150 mbar, or at least 2200 mbar.

In one aspect, the partial pressure of steam at the inlet of the physical separation device (e.g. filter) is greater than 650 mbar. In one aspect, the partial pressure of steam at the inlet of the physical separation device (e.g. filter) is at least 700 mbar, such as at least 750 mbar, at least 800 mbar, at least 850 mbar, at least 900 mbar, at least 950 mbar, at least 1000 mbar, at least 1050 mbar, at least 1100 mbar, at least 1150 mbar, at least 1200 mbar, at least 1250 mbar, at least 1300 mbar, at least 1350 mbar, at least 1400 mbar, at least 1450 mbar, at least 1500 mbar, at least 1550 mbar, at least 1600 mbar, at least 1650 mbar, at least 1700 mbar, at least 1750 mbar, at least 1800 mbar, at least 1850 mbar, at least 1900 mbar, at least 1950 mbar, at least 2000 mbar, at least 2050 mbar, at least 2100 mbar, at least 2150 mbar, or at least 2200 mbar.

In one aspect, the partial pressure of steam is no greater than 5000 mbar, such as no greater than 4500 mbar, no greater than 4000 mbar, no greater than 3800 mbar, no greater than 3600 mbar, no greater than 3400 mbar, no greater than 3200 mbar, no greater than 3000 mbar, no greater than 2800 mbar, no greater than 2600 mbar, or no greater than 2500 mbar.

In one aspect, the partial pressure of steam at the outlet of the reactor is no greater than 5000 mbar, such as no greater than 4500 mbar, no greater than 4000 mbar, no greater than 3800 mbar, no greater than 3600 mbar, no greater than 3400 mbar, no greater than 3200 mbar, no greater than 3000 mbar, no greater than 2800 mbar, no greater than 2600 mbar, or no greater than 2500 mbar.

In one aspect, the partial pressure of steam within the reactor is no greater than 5000 mbar, such as no greater than 4500 mbar, no greater than 4000 mbar, no greater than 3800 mbar, no greater than 3600 mbar, no greater than 3400 mbar, no greater than 3200 mbar, no greater than 3000 mbar, no greater than 2800 mbar, no greater than 2600 mbar, or no greater than 2500 mbar.

In one aspect, the partial pressure of steam at the inlet of the physical separation device (e.g. filter) is no greater than 5000 mbar, such as no greater than 4500 mbar, no greater than 4000 mbar, no greater than 3800 mbar, no greater than 3600 mbar, no greater than 3400 mbar, no greater than 3200 mbar, no greater than 3000 mbar, no greater than 2800 mbar, no greater than 2600 mbar, or no greater than 2500 mbar.

In one aspect, the partial pressure of steam is from 700 to 5000 mbar, such as from 750 to 4000 mbar, from 800 to 3500 mbar, from 1000 to 3000 mbar, from 1200 to 2800 mbar, from 1500 to 2700 mbar, or from 2000 to 2500 mbar.

In one aspect, the partial pressure of steam at the outlet of the reactor is from 700 to 5000 mbar, such as from 750 to 4000 mbar, from 800 to 3500 mbar, from 1000 to 3000 mbar, from 1200 to 2800 mbar, from 1500 to 2700 mbar, or from 2000 to 2500 mbar.

In one aspect, the partial pressure of steam within the reactor is from 700 to 5000 mbar, such as from 750 to 4000 mbar, from 800 to 3500 mbar, from 1000 to 3000 mbar, from 1200 to 2800 mbar, from 1500 to 2700 mbar, or from 2000 to 2500 mbar.

In one aspect, the partial pressure of steam at the inlet of the physical separation device (e.g. filter) is from 700 to 5000 mbar, such as from 750 to 4000 mbar, from 800 to 3500 mbar, from 1000 to 3000 mbar, from 1200 to 2800 mbar, from 1500 to 2700 mbar, or from 2000 to 2500 mbar.

d) Separating Solids from the Fragmentation Product

The process comprises separating solids from the fragmentation product, wherein the solids comprise solids selected from the fluidisable particles, fragments of the fluidisable particles, by-products from the thermolytic fragmentation of the feedstock solution, and mixtures thereof.

In one aspect, step d) comprises separating at least 50 wt. %, at least 60 wt. %, at least 70 wt. %, at least 80 wt. %, at least 90 wt. %, at least 95 wt. %, or at least 99 wt. % of the solids from the fragmentation product based on the total weight of the solids.

In one aspect, step d) comprises separating no greater than 99.999 wt. %, such as no greater than 99.99 wt. %, no greater than 99.9 wt. %, no greater than 99.5 wt. %, or no greater than 99 wt. % of the solids from the fragmentation product based on the total weight of the solids.

In one aspect, step d) comprises separating a major portion of the solids from the fragmentation product.

In one aspect, step d) comprises separating at least 50 wt. %, at least 60 wt. %, at least 70 wt. %, at least 80 wt. %, at least 90 wt. %, at least 95 wt. %, or at least 99 wt. % of the solids from the fragmentation product based on the total weight of the solids.

In one aspect, step d) comprises separating substantially all of the solids from the fragmentation product.

In one aspect, step d) comprises separating the solids from the fragmentation product using a physical separation device.

In one aspect, the physical separation device is a particle separator.

In one aspect, the physical separation device is a low volume separator.

In one aspect, the physical separation device is a change of direction separator.

In one aspect, the physical separation device is a cyclone.

In one aspect, the physical separation device is a filter.

In one aspect, the filter is selected from: a metal filter and a ceramic filter.

In one aspect, the filter comprises a filter element. The filter element can be considered as the material through which the fragmentation product comprising the solids (the filtration medium) is filtered, thereby to separate the solids from the fragmentation product.

In one aspect, the filter element comprises a filtration surface. The filtration surface can also be referred to as a filtration area. The filtration surface may be considered as an outer surface of the filter element. The filtration surface may be considered as the interface through which the fragmentation product comprising the solids enters the filter element in use. The filtration surface may be for separating the solids from the fragmentation product. In this way, the solids may collect on the filtration surface in use.

In one aspect, the filter element comprises a porous matrix.

In one aspect, the porous matrix is selected from; a porous fibrous matrix; a porous mesh matrix; a porous matrix comprising sintered spheres; a porous metal matrix (e.g. a sintered porous metal matrix); a porous ceramic matrix; and combinations thereof.

In one aspect, the porous matrix is a porous fibrous matrix. In one aspect, the porous fibrous matrix is a porous fibrous metal matrix. In one aspect, the porous fibrous matrix is a sintered porous fibrous metal matrix.

In one aspect, the filter comprises a plurality of the filter elements. Each of the filter elements may be independently as described herein.

In one aspect, the filter is a candle filter.

In one aspect, the filter is a bag filter.

In one aspect, step d) occurs downstream of the reactor.

In one aspect, the physical separation device is arranged downstream of the reactor.

In one aspect, multiple of the physical separation devices are employed. Each of the physical separation devices may be independently as described herein.

In one aspect, step d) comprises filtering the fragmentation product using a filter.

In one aspect, the temperature at an inlet of the filter is from 230 to 390° C., or from 270 to 390° C., from 300 to 380° C., or from 330 to 370° C.

In one aspect, the filter has an efficiency of at least 99.9% for particles having a size of at least 50 micrometres, such as at least 30 micrometres, at least 20 micrometres, or at least micrometres. The filter efficiency may be measured using ASTM 795 ACFTD.

In one aspect, the method is suitable for industrial scale production of C1-C3 oxygenates.

### The Solids

The solids comprise solids selected from the fluidisable heat carrying particles, fragments of the fluidisable heat carrying particles, by-products from the thermolytic fragmentation of the feedstock solution, and mixtures thereof.

As the fluidisable heat carrying particles are fluidised within the reactor, an amount of the particles can be transported out of the reactor, e.g. via an outlet of the reactor. The fragments of the particles can be formed by abrasion or breakage of the particles, e.g. by mechanical interaction between the particles themselves and/or between the particles and the reactor.

The by-products can be viscous and/or sticky. Without removal of the by-products, these can adhere to the filter (when present) and/or equipment downstream of the reactor. For example, when a filter is used, the by-products can form an increasingly large, sticky filter cake on the filter over time, such that, eventually, the filter cake needs to be removed.

In one aspect, the solids substantially comprise of solids selected from the fluidisable heat carrying particles, fragments of the fluidisable heat carrying particles, by-products from the thermolytic fragmentation of the feedstock solution, and mixtures thereof.

In one aspect, the solids consist essentially of solids selected from the fluidisable heat carrying particles, fragments of the fluidisable heat carrying particles, by-products from the thermolytic fragmentation of the feedstock solution, and mixtures thereof.

In one aspect, the solids consist of solids selected from the fluidisable heat carrying particles, fragments of the fluidisable heat carrying particles, by-products from the thermolytic fragmentation of the feedstock solution, and mixtures thereof.

In one aspect, the process is suitable for processing amounts of sugar of greater than 1,000 tons per year per fragmentation reactor, such as greater than 5000 tons per year per fragmentation reactor, greater than 10000 tons per year per fragmentation reactor, greater than 50000 tons per year per fragmentation reactor, greater than 100000 tons per year per fragmentation reactor, or greater than 1000000 tons per year per fragmentation reactor, based on weight of dry sugar.

In one aspect, the process is suitable for processing amounts of sugar of no greater than 10000000 tons per year per fragmentation reactor, such as no greater than 5000000 tons per year per fragmentation reactor, or no greater than 2000000 tons per year per fragmentation reactor, based on weight of dry sugar.

In one aspect, the thermolytic fragmentation method is operated as a continuous process.

### Further Process Steps

In one aspect, the process comprises one or more further separation steps between step c) and step d), wherein the or each separation between step c) and step d) comprises separating solids from the fragmentation product, wherein the solids comprise solids selected from fluidisable heat carrying particles, fragments of the fluidisable heat carrying particles, by-products from the thermolytic fragmentation of the feedstock solution, and mixtures thereof. In one aspect, the or each separation between step c) and step d) is performed within the reactor

In one aspect, the or each separation step between step c) and step d) comprises separating at least 50 wt. %, at least 60 wt. %, at least 70 wt. %, at least 80 wt. %, at least 90 wt. %, at least 95 wt. %, or at least 99 wt. % of the solids from the fragmentation product based on the total weight of the solids.

In one aspect, the or each separation step between step c) and step d) comprises separating no greater than 99.9 wt. %, such as no greater than 99.5 wt. %, no greater than 99 wt. %, no greater than 95 wt. %, no greater than 90 wt. %, or no greater than 80 wt. % of the solids from the fragmentation product based on the total weight of the solids.

In one aspect, the process comprises, between step c) and step d), a primary separation and/or a secondary separation. In one aspect, the primary separation and/or the secondary separation are performed within the reactor.

In one aspect, the primary separation comprises separating solids from the fragmentation product, wherein the solids comprise solids selected from fluidisable heat carrying particles, fragments of the fluidisable heat carrying particles, by-products from the thermolytic fragmentation of the feedstock solution, and mixtures thereof.

In one aspect, the primary separation comprises separating at least 50 wt. %, at least 60 wt. %, at least 70 wt. %, at least 80 wt. %, at least 90 wt. %, at least 95 wt. %, or at least 99 wt. % of the solids from the fragmentation product based on the total weight of the solids.

In one aspect, the primary separation comprises separating no greater than 99.9 wt. %, such as no greater than 99.5 wt. %, no greater than 99 wt. %, no greater than 95 wt. %, no greater than 90 wt. % or no greater than 80 wt. % of the solids from the fragmentation product based on the total weight of the solids.

In one aspect, the secondary separation comprises separating solids from the fragmentation product, wherein the solids comprise solids selected from fluidisable heat carrying particles, fragments of the fluidisable heat carrying particles, by-products from the thermolytic fragmentation of the feedstock solution, and mixtures thereof.

In one aspect, the secondary separation comprises separating at least 50 wt. %, at least 60 wt. %, at least 70 wt. %, at least 80 wt. %, at least 90 wt. %, at least 95 wt. %, or at least 99 wt. % of the solids from the fragmentation product based on the total weight of the solids.

In one aspect, the secondary separation comprises separating no greater than 99.9 wt. %, such as no greater than 99.5 wt. %, no greater than 99 wt. %, no greater than 95 wt. %, no greater than 90 wt. %, or no greater than 80 wt. % of the solids from the fragmentation product based on the total weight of the solids.

The process according to the present invention may provide a fragmentation product which is subjected to one or more further processing steps. For example, subsequent to step d), the fragmentation product may be further processed by one or more of chemical conversion (e.g. into another product, e.g. via a hydrogenation reactor), purification (e.g. to reduce the presence of any impurities), and separation (e.g. to provide specific oxygenates or a specific oxygenate mixture).

According to another aspect of the present invention, there is provided a system for thermolytic fragmentation of a sugar into C1-C3 oxygenates, the system comprising:
a) an aqueous feedstock solution comprising the sugar;
b) a fluidised bed fragmentation reactor configured to thermolytically fragment the sugar and comprising fluidisable heat carrying particles thereby to provide a fragmentation product comprising the C1-C3 oxygenates; and
c) a separation device configured to separate solids from the fragmentation product, wherein the solids comprise solids selected from the fluidisable heat carrying particles, fragments of the fluidisable heat carrying particles, by-products from the thermolytic fragmentation of the feedstock solution, and mixtures thereof;
wherein the temperature of the fragmentation product at an outlet of the reactor is at least 250° C., such as at least 300° C., at least 350° C., at least 400° C., or at least 450° C., and
wherein the water content is at least 40 vol. %, based on all gas phase constituents.

According to another aspect of the present invention, there is provided a system for thermolytic fragmentation of a sugar into C1-C3 oxygenates, the system comprising:
a) an aqueous feedstock solution comprising the sugar;
b) a fluidised bed fragmentation reactor configured to thermolytically fragment the sugar and comprising fluidisable heat carrying particles thereby to provide a fragmentation product comprising the C1-C3 oxygenates; and
c) a separation device configured to separate solids from the fragmentation product, wherein the solids comprise solids selected from the fluidisable heat carrying particles, fragments of the fluidisable heat carrying particles, by-products from the thermolytic fragmentation of the feedstock solution, and mixtures thereof;
wherein the temperature of the fragmentation product at an outlet of the reactor is at least 250° C., such as at least 300° C., at least 350° C., at least 400° C., or at least 450° C., and wherein the partial pressure of steam is greater than 650 mbar.

Any aspect of the present invention may comprise any feature or features of any other aspect of the present invention. The features of any aspect of the present invention may be as described herein in relation to any other aspect of the present invention. For example, the systems according to the present invention may be characterised by any of the features of the processes according to the present invention, and vice-versa.

### BRIEF DESCRIPTION OF THE DRAWINGS

Embodiments of the present invention are explained by way of examples and with reference to the accompanying drawings. The appended drawings illustrate only examples of embodiments of the present invention, and they are therefore not to be considered limiting of its scope, as the invention may admit to other alternative embodiments.

FIG. 1 shows a cross sectional side view of a fragmentation reactor which forms part of a system according to an embodiment of the invention;

FIG. 2 shows a top view of the fragmentation reactor of FIG. 1;

FIG. 3 shows a cross sectional side view of a reheater which forms part of a system according to an embodiment of the invention;

FIG. 4 shows a cross sectional side view of a system according to an embodiment of the invention (not all components of the system are shown), the system comprising a fragmentation reactor in fluid communication with a reheater;

FIG. 5 shows a block diagram of a system according to an embodiment of the invention (not all components of the system are shown);

FIG. 6 shows the variation in pressure drop across the filter of a system according to embodiment of the invention;

FIG. 7 shows a schematic diagram of a candle filter; and

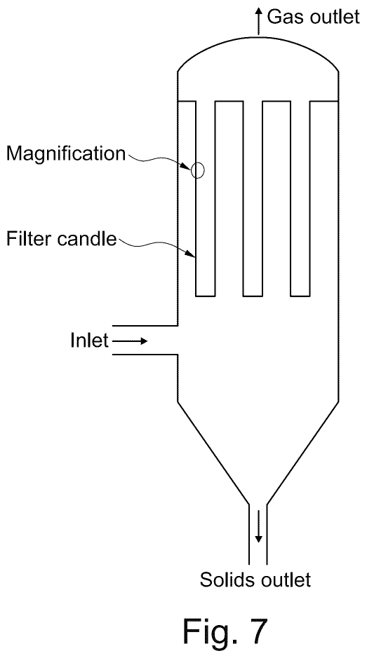

FIG. 8 shows a schematic drawing of a fragmentation reactor which forms part of a system according to an embodiment of the invention.

### POSITION NUMBERS

1. Fragmentation reactor
2. Fragmentation riser
3. First particle separator
4. Second particle separator
5. Cooling section
6. Primary fluidisation gas inlet
7. Fluidisable particle inlet
8. Feedstock and atomisation gas inlet
9. Product outlet
10. Fluidisable particle outlet
11. Reheater
12. Fuel and combustion air inlet
13. Burner chamber
14. Reheater fluidisable particle inlet
15. Reheater riser
16. Reheater fluidisable particle separator
17. Reheater fluidisable particle outlet
18. Reheater gas outlet
19. Second reheater fluidisable particle separator
20. Stripper
21. Secondary fluidisation gas inlet
22. Secondary reheater fluidisation and stripping gas inlet
O′. Outlet of the reactor
100. Heat exchanger
200. Physical separation device (e.g. filter) for gas/solid filtration
300. Fragmentation reactor
301. Fluidisation gas
302. Feedstock solution
303. Atomisation gas
304. Feedstock and atomisation gas inlet (feed nozzle)
305. First particle separator
306. Recycled heat carrying particles
307. Approximate boundary separating the dense phase and the lean phase
308. Heating arrangement

### DETAILED DESCRIPTION

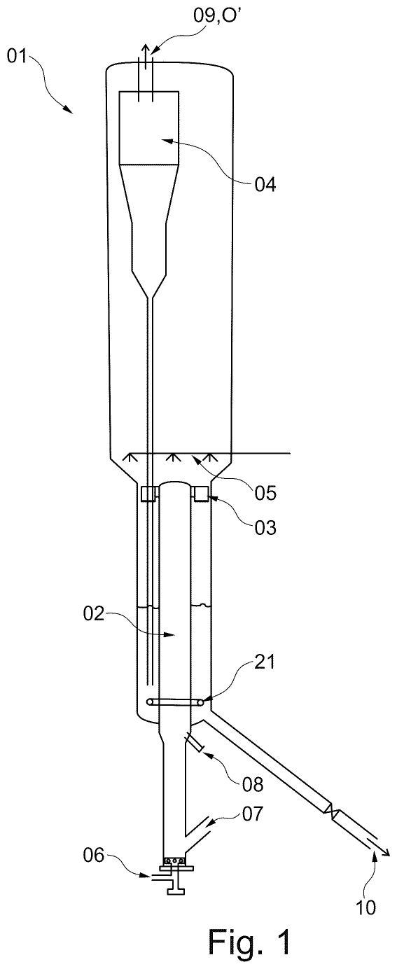

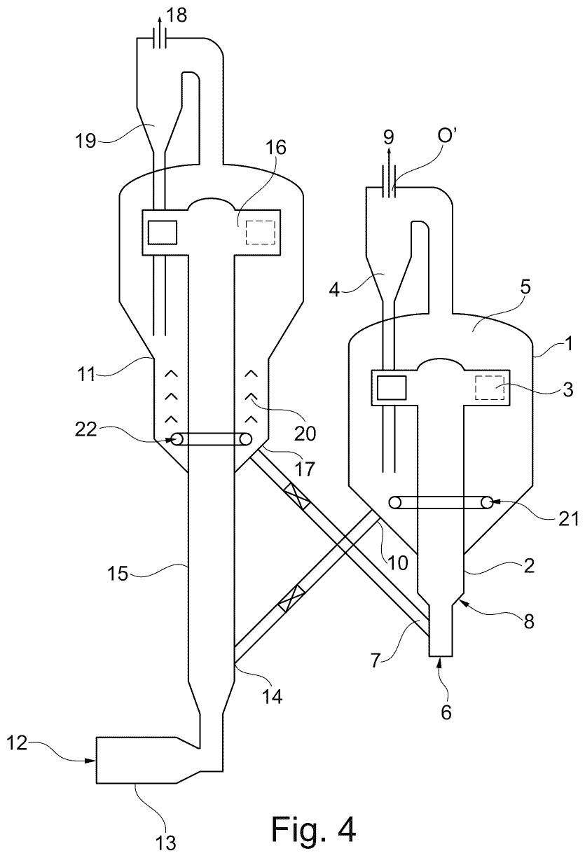

As illustrated in FIG. 1 and FIG. 4, the fragmentation reactor 01 is oblong in the vertical direction. Within the fragmentation reactor a riser 02 is provided, which is oblong with a small cross sectional area relative to the height. This facilitates the possibility of a low residence time of the fluidisable heat carrying particles inside the riser 02. In the lower section of the riser 02, a fluidisation gas inlet 06 is provided. The primary fluidisation gas inlet 06 is adapted to provide a fluidisation gas to the riser 02. In the lower section of the riser 02, a fluidisable heat carrying particles inlet 07 is also provided. The reactor 01 further comprises an outlet O′. It will be understood that, in use, the fragmentation product exits the reactor 01 at the outlet O′ for further processing and/or collection.

The fluidisation gas helps to facilitate the movement of the fluidisable heat carrying particles from the fluidisable heat carrying particle inlet 07 to the feedstock inlet 08 and towards the top of the riser 02. In addition, the fluidisation gas can be used to pre-condition the fluidisable heat carrying particles before the particles are contacted with feedstock solution.

The feedstock and atomisation gas inlet 08 is provided in the riser 02, above the particle and fluidisation gas inlets 06, 07. The feedstock inlet 08 enables the supply of a feedstock solution to the riser 02. As shown in FIG. 1, the feedstock inlet 08 is arranged in the lower section of the riser 02, although its position may vary according to the process demands.

When feedstock solution and fluidisable particles have interacted in the riser 02, they are separated when exiting the riser in a first particle separator 03. In some embodiments, the first particle separator 03 is adapted to provide a fast separation of the fluidisable particles from the fragmentation product (comprising C1-C3 oxygenates) as such a fast separation is highly advantageous to the process. Hence, the particle separator 03 can be of a low residence time type.

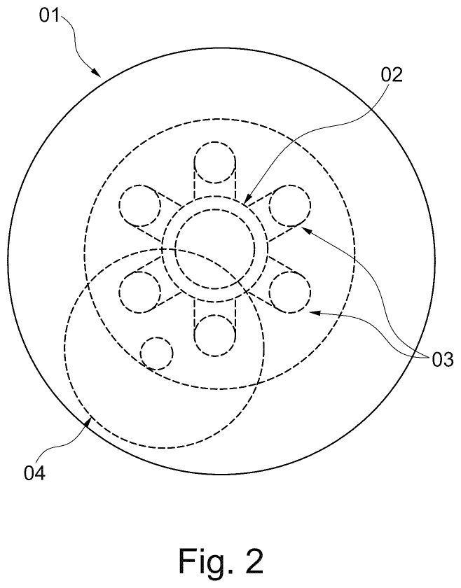

In the embodiment of FIG. 1 and FIG. 2, the first particle separator 03 comprises exit pipes which change the upwards direction of the exit flow from the riser 02 by approximately 180° into a downwards flow direction within the fragmentation reactor 01 and outside the riser 02, which in the present context is referred to as a change of direction particle separator.

In the embodiment of FIG. 4, the first particle separator 03 induces a gas particle separation, forcing a tangential, relative to the wall of reactor 01, exit of the riser gas and solids into the reactor 1 and thereby performing the separation. A portion of the particles settles at the bottom part of the fragmentation reactor 01 after exiting the first particle separator 03.

The described features of the riser 02, the position of feedstock inlet 06, and the low residence time first particle separator 03 provide the possibility of a very low contact time between fluidisable particles and feedstock solution depending, of course, also on process parameters such as volume flows and specific dimensions, which are to be adapted to the process demands.

An optional cooling section 05 is arranged within the fragmentation reactor 01. In the embodiment of FIG. 1, the cooling section is provided between the first particle separator 03 and the outlet O′ of the reactor 01.

The fragmentation product is extracted from the fragmentation reactor 01 via the product outlet 09.

In the embodiments shown in FIG. 1 and FIG. 4, an optional further second particle separator 04 is provided to separate a further fraction of the fluidisable particles from the product stream before the fragmentation product is extracted.

The second particle separator 04, such as for instance a cyclone, may present a higher separation efficiency than the change of direction separator 03 alone. The gas outlet of the cyclone 04 is connected to the product outlet 09, and the particles from the particle outlet of the cyclone 04 are carried to the bottom of the fragmentation reactor 01, where the fluidisable particles are maintained fluidised by use of fluidisation gas supplied via a secondary fluidisation gas inlet 21. The distribution of fluidisation gas over the horizontal cross section of the vessel 1 is ensured, e.g. using spargers. At the bottom of the fragmentation reactor 01, a particle outlet 10 enables the spent fluidisable particles of the fragmentation reactor 01 to be extracted and carried elsewhere, e.g. for reheating in another reactor. A stripping of product gas (fragmentation product) just before or after the fluidisable particle outlet 10 in FIG. 1-4 is also envisaged, but not shown on the figures.

FIG. 2 is a top view of the fragmentation reactor of FIG. 1. As illustrated, the riser 02 is located in the horizontal cross sectional centre of the fragmentation reactor 01. Furthermore, the plurality of exit pipes forming the first particle separator 03 is shown, as well as the second particle separator 04, which is located off-centre to the fragmentation reactor 01.

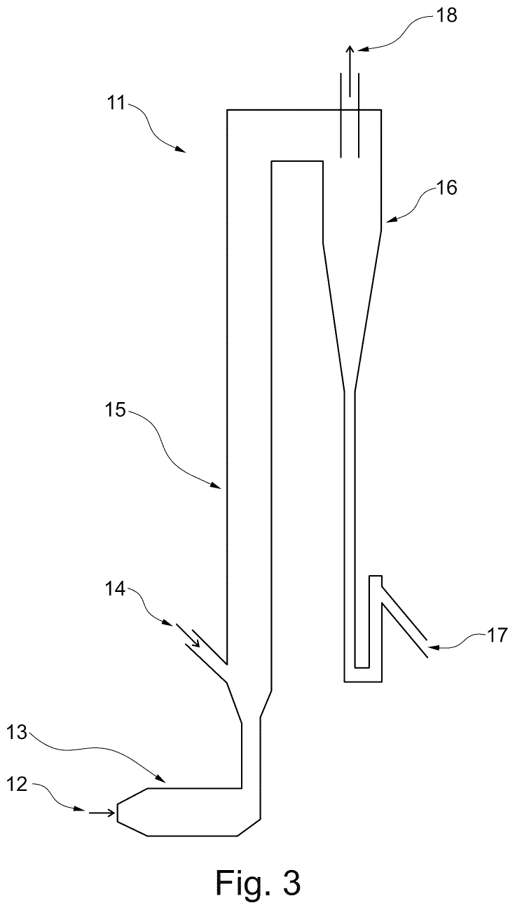

In FIG. 3 and FIG. 4, a reheater 11 for reheating the fluidisable particles exiting the fragmentation reactor 01 is shown. The reheater particle inlet 14 is in fluid connection with the fluidisable particle outlet 10, and the reheater fluidisable particle outlet 17 is in fluid connection with the fluidisable particle inlet 07. The reheater 11 also comprises a riser type fluidised bed, and a reheater riser 15, with a burner chamber 13 arranged in fluid connection to the lower part of the reheater riser 15. A fuel and combustion air inlet 12 enables fuel and combustion air to be provided to the burner chamber 13, which, during operation, provides heat to the reheater riser 15. The reheater fluidisable particle inlet 14 is arranged in the lower part of the reheater riser 15 and enables the fluidisable particles exiting the fragmentation reactor 01 to enter the reheater riser 15 where they are fluidised in an upwards flow by the hot gas provided by the burner chamber 13 while being heated. The connection between the burner chamber 13 and the reheater fluidisable particle inlet 14 is deliberately designed to reduce or prevent fall-through of fluidisable particles from the riser 15 into the burner chamber 13. This design could take many different forms. For example, in FIG. 3 and FIG. 4, this is illustrated by the constriction between 13 and 15 leading to an increased gas velocity preventing or reducing a fall-through of particles. After reheating, the fluidisable particles are separated from the combustion gas and are fed back to the fragmentation reactor 01. In the embodiment of FIG. 3, the reheater particle separator 16 is a cyclone which enables gas to exit the reheater 11 via the reheater gas outlet 18 while the separated fluidisable particles exit the reheater 11 via the reheater fluidisable particle outlet 17 connected to the fluidisable particle outlet of the cyclone of the reheater 11. It is to be understood that the extent of separation in the particle separators depends on various process parameters, such as pressure loss in the separator, flow velocities, particle size etc., as known in the art.

In the embodiment of FIG. 4, the reheater first particle separator 16 is similar to the fragmentation reactor first particle separator 03. The embodiment of FIG. 4 is also equipped with a secondary cyclone type particle separator 19. Both particle separators 16, 19 deliver fluidisable particles to the bottom of reheater 11. In the lowermost position of the reheater 11, a section 20 for stripping excess oxygen from the fluidisable particles is placed. Secondary fluidisation and stripping gas inlets 22 for the reheater 11 and the section 20 in the embodiment of FIG. 4 are distributed over the cross section using e.g. spargers or other methods. Additional fluidisation gas inlets may be present in the reheater 11 in FIG. 4, but are not shown. A stripping of product gas (fragmentation product) just before or after the fluidisable particle outlet 10 in FIG. 1-4 is also envisaged, but not shown on the figures.

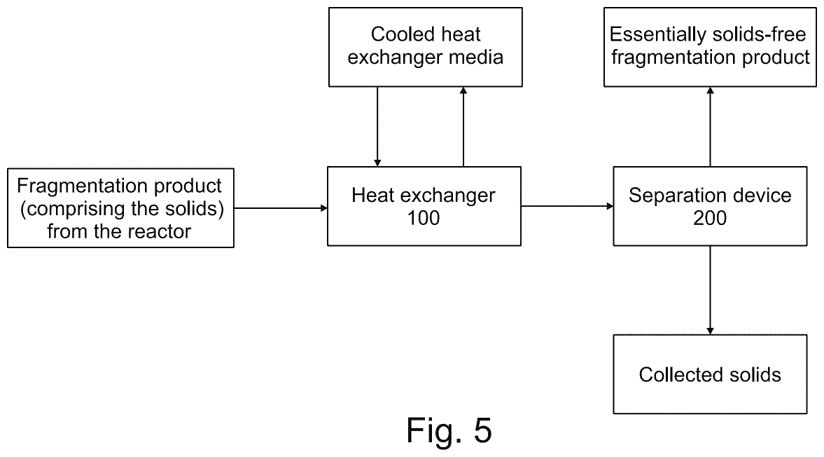

With reference to FIG. 5, further components of the system of FIG. 4 are illustrated (although not all components of the system are illustrated). The system comprises the components of FIG. 4. In other embodiments, the system includes the fragmentation reactor of FIG. 1 instead of that shown in FIG. 4. The system may further comprise a cooling device, such as a heat exchanger 100, for cooling the product gas (fragmentation product). The system also comprises a physical separation device 200 (discussed below). In the embodiment of FIG. 5, the heat exchanger 100 is arranged downstream of the reactor 1, and the physical separation device 200 is arranged downstream of the heat exchanger 100. Those skilled in the art will understand that the system may comprise further components, such as one or more cooling steps or other unit operations.

In the embodiment of FIG. 5, the product gas traverses the product outlet 9 to the heat exchanger 100, and then to the physical separation device 200, and then to further downstream equipment. In the embodiment of FIG. 5, the physical separation device 200 is a filter, specifically a candle filter wherein the filter element provided by the candles of the candle filter comprised a porous matrix of metal fibres. More specifically, the metal fibres comprised a nickel-chromium-molybdenum alloy (alloy 59).

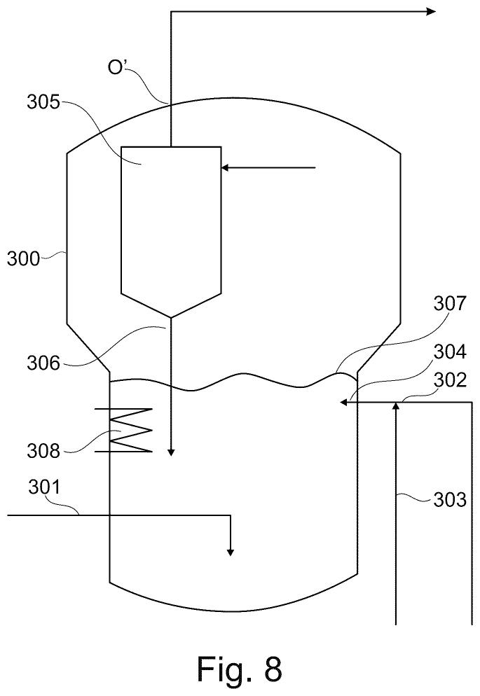

FIG. 8 shows a schematic diagram of the candle filter. The candle filter comprises an enclosure having a solids/fragmentation product inlet through which the fragmentation product comprising the solids are directed, a solids outlet through which separated solids are directed, and a fragmentation product/gas outlet through which the fragmentation product from which solids have been separated is directed. A plurality of candles are arranged within the enclosure. Each candle comprises a filter element for separating the solids from the fragmentation product. Each filter element comprises a filtration surface (or filtration area). The filtration surface may be considered as the interface through which the fragmentation product comprising the solids enters the filter element in use. The filtration surface is the outer surface of the filter element. In use, the fragmentation product enters each filter element via the filtration surfaces, such that the solids are separated from the fragmentation product by each filter element. The separated solids are removed from the enclosure via the solids outlet. The solids outlet may be equipped with a valve system (not shown) to prevent gas from escaping and to remove the collected solids.

In other embodiments, other physical separation devices and other filters are envisaged. For example, candle filters are described in Gasification (Higman, C., 2nd edition, 2008, p. 224-225). The functioning and operation of a candle filter is known to those skilled in the art, and therefore is not described herein in further detail.

In use, the fragmentation product exits the reactor 01 via the product outlet 09 and passes through the heat exchanger 100 before passing through the separation device 200.

The present inventors surprisingly discovered the presence of material adhered to equipment downstream of the reactor 01. In some instances, this material resulted in obstruction or blockage in the equipment. The present inventors conducted experiments to characterise the material, and identified that such material comprised solids selected from fluidisable heat carrying particles (transported from the reactor 01), fragments of the fluidisable heat carrying particles (transported from the reactor 01), by-products from the thermolytic fragmentation of the feedstock solution, and mixtures thereof. The present inventors tested various means (e.g. the physical separation device 200 such as a filter) for reducing the amount of the solids transported downstream of the reactor 01. Whilst such means initially helped to achieve this, the present inventors further discovered that the solids deposited onto such means (e.g. as “filter cake”) in an increasing amount as a function of time (reactor run time). So as to facilitate effective process operation, the solids eventually were removed. However, removal of the solids by replacement of the means involves shutting down the process, resulting in significant downtime which is inefficient. Where the means comprised a filter 200, the present inventors also attempted to remove the solids from the filter 200 by “back-pulsing” or “back-blowing” techniques, e.g. which involved directing air in a reverse direction through the filter 200. However, these techniques were not effective because the solids remained adhered to the filter 200.

With the above problems in mind, the present inventors surprisingly discovered that the presence of water in an amount of at least 40 vol. % based on all gas phase constituents resulted in an improved performance of equipment downstream of the reactor 01. In particular, such a water content resulted in reduced obstruction and/or blockage in equipment downstream of the reactor 01. Furthermore, where a physical separation device such a filter 200 arranged downstream of the reactor 01 was employed, the present inventors surprisingly also discovered that such a water content reduced the build-up of the solids on the physical separation device 01.

Without being bound by theory, it is hypothesised that such a water content reduces the amount of the solids that are transported downstream of the reactor 01 and/or reduces the propensity of the solids to form and/or reduces the propensity of the solids to obstruct or block the equipment downstream of the reactor 01. Moreover, when a physical separation device such a filter 200 arranged downstream of the reactor is employed, it is further hypothesised that such a water content reduces the amount of the solids that reach the physical separation device 200 and/or reduces the propensity of the solids to adhere to the physical separation device 200 and/or reduces the propensity of the solids to obstruct or block the physical separation device 200.

For example, in embodiments comprising a filter 200 arranged downstream the reactor 01, using steam instead of nitrogen, e.g. provided as atomisation gas via inlet 8 and/or as fluidisation gas via inlets 6, 21, can lead to a surprisingly beneficial effect on the observed filter pressure drop during operation. That is, water can be used to maintain a more level pressure drop across the filter 23 during operation. By contrast, when nitrogen or indeed a low concentration of water is used, the observed filter pressure drop progressively increases during operation. The beneficial effect on the observed pressure drop indicates a reduction in the amount of the solids being transported downstream the reactor 1 and collecting on the filter 200. As such the process can be operated for a longer duration before replacement of the filter 200 is required.

As will be understood by those skilled in the art, the water may be introduced into the reactor by various means, including, for example, via the feedstock solution, the fluidisation gas, and/or the atomisation gas. However, it is to be understood that the fluidisation gas and/or the atomisation gas may comprise inert gases, such as N₂, CO, CO₂, CH₄, or mixtures thereof.

### Example 1: The Effect of Water Content on the Pressure Drop Across the Filter

Thermolytic fragmentation of an aqueous feedstock solution comprising approximately 60 wt. % glucose and 40 wt. % water (based on the total weight of the feedstock solution) was conducted in a system according to an embodiment of the invention (i.e. FIGS. 4 and 5). This involved introducing the feedstock solution into the reactor 01 at a (liquid) feed rate of 30 kg per hour at position 8. The fragmentation was performed under the following conditions:

(A) A water content of 35-40 vol. % based on all gas phase constituents immediately before the first particle separator (3) and a water content of 25-30 vol. % based on all gas phase constituents at the outlet O′ of the reactor 01 (steam partial pressure of 880 to 1030 mbar). The reactor was a riser type reactor 01 having a length of approximately 3 meters and a superficial gas velocity of approximately 6 meters per second after evaporation and fragmentation of the feedstock solution. The feedstock solution was injected into the reactor 1 using the gas assisted internal mix atomisation nozzle (i.e. the feedstock and atomisation gas inlet 08) entering from the base of the riser 02, with an atomisation gas flow of 15 kg/h of nitrogen. The optional cooling step 05 was omitted.

(B) A water content of 80-81 vol. % based on all gas phase constituents immediately before the first particle separator (3) and a water content of 85-90 vol. % based on all gas phase constituents at the outlet O′ of the reactor 01 (steam partial pressure of 2040 mbar). The reactor was a riser type reactor 01 having a length of approximately 3 meters and a superficial gas velocity of approximately 6 meters per second after evaporation and fragmentation of the feedstock solution. The feedstock solution was injected into the reactor 1 using the gas assisted internal mix atomisation nozzle (i.e. the feedstock and atomisation gas inlet 08) entering from the base of the riser 02, with an atomisation gas flow of 12 kg/h of steam. The optional cooling step 05 was activated from 15 run hours to 25 run hours, and cooled the product gas by approximately 50° C.

For (A), nitrogen was introduced to the reactor via: the fluidisation inlet 6 at a flow rate of 8.7 kg/h of nitrogen; the feedstock and atomisation gas inlet 8 (i.e. as atomisation gas added with the feedstock solution, as discussed above); and the fluidisation gas inlet 21 at a flow rate of 15 kg/h of nitrogen. The total pressure measured at the outlet O′ of the reactor 01 was approximately 2.4 bara (absolute pressure). The temperature of the fluidisable heat carrying particles at inlet 07 was approximately 570° C. and the temperature at the outlet O′ of the reactor was approximately 475° C. The temperature at an inlet to the filter 200 was approximately 400° C.

For (B), water was introduced to the reactor via: the fluidisation inlet 6 at a gas flow rate of 7 kg/h of steam; the feedstock and atomisation gas inlet 8 (i.e. as atomisation gas added with the feedstock solution, as discussed above); and the fluidisation gas inlet 21 at a flow rate of 12 kg/h of steam. The total pressure measured at the outlet O′ of the reactor 01 was approximately 2.4 bara (absolute pressure). The temperature of the fluidisable heat carrying particles at inlet 07 was approximately 570° C. and the temperature at the outlet O′ of the reactor was approximately 475° C. The temperature at an inlet to the filter 200 was approximately 400° C.

For (B), the mass ratio of the total amount of sugar introduced into the reactor 01 to the total amount of water introduced into the reactor was 0.41:1.

The gas flows on volumetric basis in the system were similar in the two cases, providing the same superficial gas velocities and residence times in the different parts of the system.

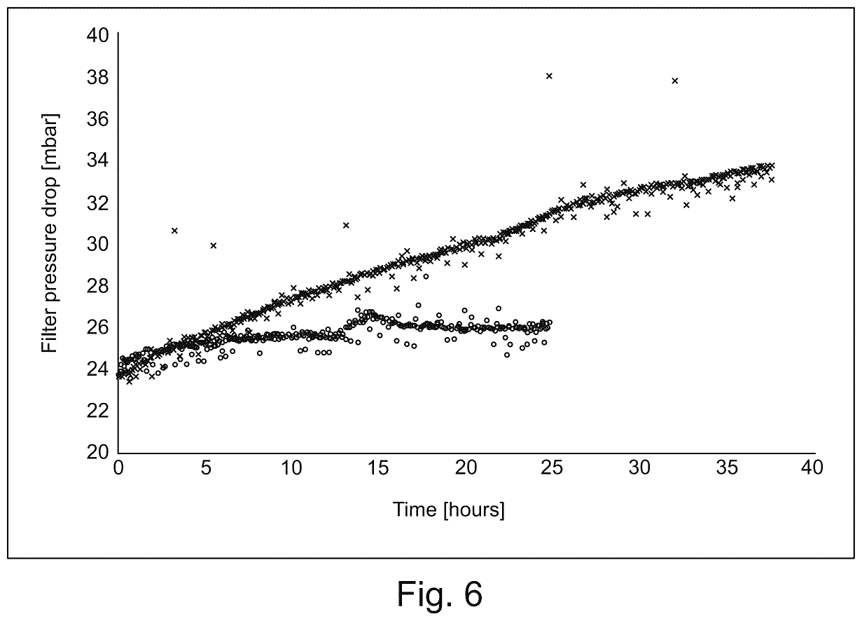

FIG. 6 shows the variation in measured pressure drop across the filter 200 during operation for (A)—cross data points and (B)—circular data points. The water content at the outlet O′ of the reactor 01 was calculated using the flow data of the input streams to the reactor 01. Those skilled in the art can readily perform this calculation.

As is apparent from FIG. 6, for (A) the pressure drop across the filter 200 steadily increased over time at a rate of approximately 0.27 mbar/hour. As is also apparent from FIG. 6, for (B) the pressure drop across the filter 200 remained approximately constant (rate of change of approximately 0 mbar/hour). This is a significantly improved performance parameter for industrial long term operation. The slight change in measured pressure drop for (B) at around 15 hours was due to the water content being increased from 80 vol. % to 81 vol. % (based on all gas phase constituents immediately before the first particle separator (3)) and a higher gas flow.

It is to be understood that the filter pressure drop is also a function of filtration parameters such as the surface area of the filter 200 (which in this example was 3.4 m² at the gas inlet surface of the filter), the nature of the gas flows (e.g. flow rates and composition), and the thickness of the filter cake (comprising the solids) deposited on filter 200. For example, increasing the surface area of the filter or decreasing the gas flow rate can reduce the rate of increase of pressure drop during operation. It also is to be understood that the reactor could be operated in another non-intended mode which produces even more fragmentation by-products. Those skilled in the art would know how to control the commonly known effect of filtration parameters.

This example indicated that an increase in water content, e.g. at the outlet O′ of the reactor 01, reduced the amount of the solids transported downstream the reactor and collected on the separation device 200. Without being bound by theory, it is believed that such a water content may reduce the rate of deposition of the solids on the separation device 200 and/or increase the rate of desorption of the solids from the separation device 200. For example, the water content may be such that the net build-up of the solids on the separation device 200, i.e. the deposition rate minus the desorption rate, may be reduced relative to when lower water contents are used.

In each of (A) and (B), following filtration at the filter 200, a solids-lean fragmentation product was obtained. The solids-lean fragmentation product was essentially free of the solids.

In each of (A) and (B), following the filtration at the filter 200, the stream of fragmentation product (solids-lean) was condensed in a cooling tower with recirculated condensed product liquid. The glycolaldehyde concentration of the condensed product (solids-lean) was measured by HPLC and a mass (or carbon) balance was applied to derive the carbon-based yield of glycolaldehyde from the thermolytic fragmentation, i.e. the percentage of carbon in the feedstock solution that is recovered as carbon in glycolaldehyde. For example, the glycolaldehyde concentration of the condensed product (solids-lean) can be calculated according to the following formula:

Glycolaldehyde carbon yield=100%*(2*mc*Cc/(MwGA*DensC))/(6*m1*C1/MwGlu)
Where:
mc is measured mass flow in kg/h out of condensation step
Cc is glycolaldehyde concentration in kg/m3 out of condensation step
MwGA is the molecular weight of glycolaldehyde in kg/mol
DensC is the density of the condensed product in kg/m3
m1 is the feedstock solution feed rate into the reactor 01 in kg/h
C1 is the glucose concentration in wt. %
MwGlu is the molecular weight of glucose in kg/mol

The resulting glycolaldehyde carbon yield for (A) was approximately 48%. The resulting glycolaldehyde carbon yield for (B) was approximately 53%. Those skilled in the art will know how to calculate the glycolaldehyde carbon yield.

### Example 2

This example illustrates another aspect of performing thermolytic fragmentation of sugars in an industrial setting according to the invention. Referring to FIG. 8, the system and process uses a fluidised bed fragmentation reactor (300). Inside the reactor (300), a fluidisation gas (301) is added by a gas distributor (not shown in detail) in the bottom of the reactor 300, for fluidisation of heat carrying particles used for thermolytic fragmentation of the sugars. The gas distributor distributes the fluidisation gas (301) over the entire cross section of the reactor (300). In FIG. 8, the gas distributor is a gas sparger having a downward direction (although other directions such as upward, horizontal or therebetween are also a possibility). Liquid sugar feedstock (302) is mixed with atomisation gas (303) in the feed nozzle (304) to provide a fine spray of liquid droplets before contacting the heat carrying particles in the reactor (300). Only one feed nozzle (304) is shown in FIG. 8 but it is to be understood that multiple feed nozzles can be used and placed at different positions within the reactor (300) to provide a good distribution of feed (302) over the reactor (300).

Liquid sugar feedstock (302) thus brought into contact with the heat carrying particles undergo cracking within the reactor and a gas phase C1-C3 oxygenate rich fragmentation product is obtained. The cracking and feed water evaporation increases the gas volume flow thus increasing the upward gas flow rate. The gas flows upwards and into a cyclone (305) (the first particle separator). Inside the cyclone (305), most (e.g. at least 95 wt. %) of the entrained heat carrying particles are separated and recycled (306) to the lower part of the reactor (300). The outlet of the reactor O′ conveys the fragmentation product gas to downstream processing. Only one particle separator (305) is shown but it is to be understood that the particle separator can be configured in different ways and multiple particle separators may be used. For example, parallel cyclones, or cyclones in series, or a combination thereof may be used. A surface filtration filter may also be used.

The lower part of reactor (300) operates in a dense type of fluidised bed with superficial gas velocities below approximately 2 m/s (meters per second). The upper part of reactor 300 operates in a lean type of phase with superficial gas velocities above approximately 3 m/s. The demarcation between the mode of operation (dense or lean) is not sharp cut and may also be dependent on gas and solid physical properties such as densities, gas viscosity and particle size and shape. The increase in gas flow rate from the cracking process may also depend on feed concentration and product yields. In FIG. 8, the approximate boundary separating the dense phase and the lean phase is indicated by the curved line (307). The curved line (307) also shows the approximate position of the top surface of the dense fluidised bed of heat carrying particles.

Inside the dense fluidised bed of heat carrying particles, an indirect heating arrangement (308) is positioned to bring the energy required for the thermolytic fragmentation step and to account for heat losses and product gas losses. The heating arrangement (308) could be resistive electrical heating rods or other types or indirect heating methods. The dense mode of fluidsation ensures good mixing and transport of the heat carrying particles in the reactor (300).

The heating arrangement (308) could also be positioned externally to reactor (300), and additional means for transferring of the heat carrying particles between the reactor (300) and the heating arrangement (308) may be provided. This configuration is not shown.

The system of FIG. 8 may be used for performing thermolytic fragmentation of sugars under industrial conditions. To the reactor (300) is added approximately 25000 liters (bulk) of heat carrying particles as described in Example 2 of WO 21/032590. An aqueous solution of 60 wt. % glucose (302) is fed into the reactor (300) at a rate of 23000 kg/h (kilograms per hour). Steam as the atomisation gas (303) is fed into the reactor (300) at a rate of 1380 kg/h. Steam as the fluidisation gas (301) is fed into the reactor (300) at a rate of 5741 kg/h. The following yields of mainly C2-C3 oxygenates on carbon basis from glucose are obtained: 57 C-% glycolaldehyde, 10 C-% pyruvaldehyde, 4 C-% acetol, 3 C-% glyoxal, and 10 C-% formaldehyde.

The water content based on all gas phase constituents at the outlet O′ of the reactor (300) is above approximately 75 vol. %. Yields of other side products, such as CO, CO2, acetic acid may not change this steam volume fraction significantly. It is to be understood that, for example, varying the flow rate and/or composition of the atomisation gas (303) or fluidisation gas (301) may change the resulting steam volume fraction at the outlet O′.

Those skilled in the art will appreciate that the present invention may be scaled-up as desired. The present invention is suitable for industrial scale production.

Various modifications and variations of the present invention will be apparent to those skilled in the art may be introduced without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A process for thermolytic fragmentation of a sugar into C1-C3 oxygenates, the process comprising the steps of:
a) providing an aqueous feedstock solution comprising the sugar;
b) providing a fluidised bed fragmentation reactor for thermolytically fragmenting the sugar and comprising fluidisable heat carrying particles;
c) introducing the feedstock solution into the reactor to thermolytically fragment the sugar to provide a fragmentation product comprising the C1-C3 oxygenates; and
d) separating solids from the fragmentation product, wherein the solids comprise solids selected from the fluidisable heat carrying particles, fragments of the fluidisable heat carrying particles, by-products from the thermolytic fragmentation of the feedstock solution, and mixtures thereof;
wherein the temperature of the fragmentation product at an outlet of the reactor is at least 250° C., and
wherein the water content is at least 40 vol. %, based on all gas phase constituents.

2. The process according to claim 1, wherein the water content at the outlet of the reactor is at least 40 vol. %, based on all gas phase constituents at the outlet of the reactor.

3. The process according to claim 1, wherein the partial pressure of steam at the outlet of the reactor is greater than 650 mbar.

4. The process according to claim 1, wherein the water content at the outlet of the reactor is at least 60 vol. %, based on all gas phase constituents at the outlet of the reactor.

5. The process according to claim 1, wherein step d) comprises separating the solids from the fragmentation product using a physical separation device.

6. The process according to claim 5, wherein the physical separation device is arranged downstream of the reactor.

7. The process according to claim 5, wherein the physical separation device is a filter.

8. The process according to claim 5, wherein the filter comprises a filter element having a filtration surface.

9. The process according to claim 5, wherein the water content is based on all gas phase constituents at an inlet of the physical separation device.

10. The process according to claim 1, comprising introducing an atomisation gas into the reactor.

11. The process according to claim 1, wherein the sugar is a carbohydrate comprising one or more C6 saccharide units and/or C5 saccharide units, and/or wherein the sugar is a monosaccharide or a disaccharide, and/or wherein the sugar is selected from sucrose, lactose, xylose, arabinose, ribose, mannose, tagatose, galactose, glucose, and fructose.

12. The process according to claim 1, wherein the total sugar content in the feedstock solution is from 30 to 99 wt. %, based on the total weight of the feedstock solution.

13. The process according to claim 1, wherein the fragmentation product comprises glycolaldehyde.

14. The process according to claim 1, wherein the fragmentation product comprises glycolaldehyde in an amount of at least 10 wt. % based on the total weight of the fragmentation product.

15. The process according to claim 1, wherein the pressure at the outlet of the reactor is at least 0.9 bara.

16. The process according to claim 1, comprising one or more further separation steps between step c) and step d), wherein the or each separation between step c) and step d) comprises separating solids from the fragmentation product, wherein the solids comprise solids selected from fluidisable heat carrying particles, fragments of the fluidisable heat carrying particles, by-products from the thermolytic fragmentation of the feedstock solution, and mixtures thereof, wherein the or each separation step between step c) and step d) comprises separating at least 50 wt. % of the solids from the fragmentation product based on the total weight of the solids.

17. A system for thermolytic fragmentation of a sugar into C1-C3 oxygenates, the system comprising:
a) an aqueous feedstock solution comprising the sugar;
b) a fluidised bed fragmentation reactor configured to thermolytically fragment the sugar and comprising fluidisable heat carrying particles thereby to provide a fragmentation product comprising the C1-C3 oxygenates; and
c) a separation device configured to separate solids from the fragmentation product, wherein the solids comprise solids selected from the fluidisable heat carrying particles, fragments of the fluidisable heat carrying particles, by-products from the thermolytic fragmentation of the feedstock solution, and mixtures thereof;
wherein the temperature of the fragmentation product at an outlet of the reactor is at least 250° C., and wherein the water content is at least 40 vol. %, based on all gas phase constituents.

18. The system according to claim 17, wherein the water content at the outlet of the reactor is at least 40 vol. %, based on all gas phase constituents at the outlet of the reactor.
